(19) United States
(12) Patent Application Publication
(10) Pub. No.: US 2023/0119794 A1
(43) Pub. Date: Apr. 20, 2023

(54) Hydrophilic Fluororesin Tube and Method for Manufacturing Hydrophilic Fluororesin Tube

(71) Applicant: Valqua, Ltd., Tokyo (JP)

(72) Inventors: Yoshihiro Setoguchi, Tokyo (JP); Naoki Watanabe, Tokyo (JP); Hirotaka Muto, Tokyo (JP)

(21) Appl. No.: 17/966,287

(22) Filed: Oct. 14, 2022

(30) Foreign Application Priority Data
Oct. 15, 2021 (JP) 2021-169486

(51) Int. Cl. D04H 1/4318; D06M 15/333; D04H 1/724; D04H 1/4382; D06M 15/327; F16L 11/02; C12N 5/00

(52) U.S. Cl. CPC D04H 1/4318; D06M 15/333; D04H 1/724; D04H 1/43838; D06M 15/327; F16L 11/02; C12N 5/0068; D06M 2101/22

(57) ABSTRACT

A hydrophilic fluororesin tube includes a tube wall that contains a fluororesin fiber deposited to form a nonwoven fabric, and satisfies the following requirement (1): requirement (1); the tube wall of the hydrophilic fluororesin tube demonstrating an average transmittance, when immersed in water at 25° C. for 1 minute, of 40% or larger at 400 to 700 nm wavelength.

### CROSS-REFERENCE TO RELATED APPLICATION

This application claims priority to Japanese Patent Application No. 2021-169486 filed Oct. 15, 2021, the disclosure of which is hereby incorporated by reference in its entirety.

### BACKGROUND OF THE INVENTION

### 1. Field of the Invention

An embodiment of the present invention relates to a hydrophilic fluororesin tube, or a method for manufacturing a hydrophilic fluororesin tube.

### 2. Description of the Related Art

In recent years, articles having porous structure, such as cell culture support, have been used in the fields of, for example, medicine and biology. The cell culture support having porous structure has been developed, typically in the form of porous membrane made of a fluorine-containing resin, having a large number of pores with a pore size of 0.5 to 10 μm (JP 2016-214210 A, for example).

With use of surface of a cylindrical tube made of such porous membrane or inside of such porous membrane as a scaffold member, for example, microorganism or biological tissue (cell, for example) has been placed or immobilized, for culturing or differentiation.

In recent years, also a tube (reactor) has been used as a reaction field of flow reaction, in which reaction proceeds under flow of raw materials.

### SUMMARY OF THE INVENTION

For example, microorganism or biological tissue, when placed or immobilized for, for example, the culturing on the cylindrical tube surface or inside of the porous membrane as described above, is necessarily observed regarding the state of progress. The reaction would be necessarily monitored also during the flow reaction.

The prior tube having been used for such purpose needs be highly hydrophilic. An object to be observed on the tube surface or inside the porous membrane (referred to as “tube content”, hereinafter) has been observable only after being taken out from the tube. Recent needs are particularly focused on observation of the tube content, while keeping it inside the tube. There has, however, been no tube allowed for such observation.

An embodiment of the present invention is to provide a hydrophilic fluororesin tube allowed for observation of the tube content while kept in water.

Exemplary configurations of the present invention are as follows.

[1] A hydrophilic fluororesin tube having a tube wall that includes a fluororesin fiber deposited to form a nonwoven fabric, and satisfying the following requirement (1).

Requirement (1): the tube wall of the hydrophilic fluororesin tube demonstrates an average transmittance, when immersed in water at 25° C. for 1 minute, of 40% or larger at 400 to 700 nm wavelength.

[2] The hydrophilic fluororesin tube according to [1], wherein the tube wall has a thickness of 10 to 150 μm.

[3] The hydrophilic fluororesin tube according to [1] or [2], wherein the tube wall has a Gurley permeability of 0.5 to 100 s, when measured in a direction perpendicular to the wall face.

[4] The hydrophilic fluororesin tube according to any one of [1] to [3], including the tube wall that contains a polytetrafluoroethylene (PTFE) fiber.

[5] The hydrophilic fluororesin tube according to [4], wherein the PTFE fiber is a PTFE fiber coated with at least one polymer compound selected from polyvinyl alcohol and modified product thereof, polysaccharide and derivative thereof, collagen, gelatin, copolymer of vinyl alcohol and vinyl group-containing monomer, polyol, polyurethane, methacrylate and modified product thereof, and hydroxy group-containing (meth)acrylic polymer.

[6] The hydrophilic fluororesin tube according to [5], wherein the polymer compound has at least one functional group selected from hydroxy group, carboxylic acid group, sulfonic acid group, phosphonic acid group, ether group, epoxy group, amino group, amido group, and quaternary ammonium salt.

[7] A method for manufacturing a hydrophilic fluororesin tube, the method including:

(1) spinning a fiber-forming material that contains at least one kind of fluororesin;

(2) forming a fluororesin tube by depositing the fiber obtained in the step (1) onto a mandrel collector; and

(3) hydrophilizing the fluororesin tube obtained in the step (2).

[8] The method for manufacturing a hydrophilic fluororesin tube according to [7], wherein

the fluororesin is PTFE, and

the step (2) is a step for forming a fluororesin tube by depositing the fiber obtained in the step (1), and then by firing the deposit.

An embodiment of the present invention can provide a hydrophilic fluororesin tube that allows, for its light transmissivity, the tube content to be observed (under an optical microscope, for example) in an aqueous medium, and that excels in chemical resistance against aqueous solution containing, for example, acid or alkali, or against organic solvent such as alcoholic solvent or amphiphilic solvent, thus enabling suitable use for applications where such functionality is required, such as culture tube or a culture medium typically for culturing, for example, microorganism or biological tissue (cell, for example), and reaction field of flow reaction.

An embodiment of the present invention can also provide a hydrophilic fluororesin tube that excels in chemical resistance against aqueous solution containing, for example, acid or alkali, or against organic solvent such as alcoholic solvent or amphiphilic solvent, thus enabling separation of a target substance, while observing (visually recognizing, for example) the tube content that presents in a center cavity of the tube in various media. Hence, the hydrophilic fluororesin tube is also suitably used as a separation tube (filter). The hydrophilic fluororesin tube according to an embodiment of the present invention, when used for separation of bio-related substance, suitably suppresses adsorption of, for example, biological substance (such as exosome) or biological polymer (such as protein).

### BRIEF DESCRIPTION OF THE DRAWINGS

FIG. 1 is a schematic drawing of a hydrophilic fluororesin tube according to an embodiment of the present invention;

FIG. 2 is a SEM image of the fluororesin tube obtained in Example 1 (before hydrophilized), at a magnification of 5000×;

FIG. 3 is a SEM image of the fluororesin tube obtained in Example 1 (before hydrophilized), at a magnification of 1000×; and

FIG. 4-left is a photograph of an appearance of the hydrophilic fluororesin tube obtained in Example 1 before immersion in water, and FIG. 4-right is a photograph of an appearance of the hydrophilic fluororesin tube obtained in Example 1, immediately after immersion in water conducted similarly to the later-described average transmittance test under water immersion.

### DESCRIPTION OF THE INVENTION

<<Hydrophilic Fluororesin Tube>>

A hydrophilic fluororesin tube according to an embodiment of the present invention (also referred to as “the present tube”, hereinafter) is a tube having a tube wall that includes a fluororesin fiber deposited to form a nonwoven fabric, and satisfies the following requirement (1).

Requirement (1): the tube wall of the present tube demonstrates an average transmittance at 400 to 700 nm wavelength, when immersed in water at 25° C. for 1 minute (also referred to as “average transmittance under water immersion”), of 40% or larger.

The average transmittance under water immersion of the present tube is preferably 50% or larger, and more preferably 60% or larger, from the viewpoint, for example, that the tube content is easily observable in water.

The average transmittance under water immersion is specifically measured by a method described later in EXAMPLES.

The present tube also preferably demonstrates the average transmittance under water immersion within the aforementioned range, when measured with (near) ultraviolet or (near) infrared radiation. More specifically, the average transmittance at 220 to 1000 nm wavelength preferably falls within the aforementioned range, when immersed in water is the same way as described above.

The tube wall that constitutes the present tube preferably has a contact angle (25° C.) for water, measured 10 seconds after dropping thereon 1 μL of water, of 30° or smaller, which is more preferably 10° or smaller. The smaller the lower limit value, the better, wherein the contact angle is typically 0°.

With the contact angle for water fallen within the aforementioned range, the tube content is easily observable.

The contact angle for water of 0° means that the dropped water is absorbed within 10 seconds after dropping 1 μL of water, with no water droplet remained on the tube wall (complete wetting).

The contact angle for water is specifically measured by a method described later in EXAMPLES.

Although the average transmittance under water immersion and the contact angle for water are values measured with water (pure water), the present tube preferably demonstrates such values of the average transmittance and the contact angle, even if any different aqueous medium were used depending on applications.

The aqueous medium refers to a liquid that contains water as a major component (more than 50% by mass) of solvent or dispersion medium, and is specifically exemplified by physiological saline, various buffers (phosphate buffers, for example), aqueous solutions containing at least one selected from acid, alkali or alcohol, and culture solution for, for example, microorganism or biological tissue.

The aqueous medium preferably has a pH of 5 to 12, which is more preferably 6 to 8.

The present tube is not particularly limited as long as it is a tube constituted by a tube wall that includes a fluororesin fiber deposited to form a nonwoven fabric, which is specifically a tube constituted by the tube wall that includes a hydrophilized fluororesin fiber deposited to form a nonwoven fabric, and is more preferably a tube constituted by the tube wall (solely) made of a hydrophilized fluororesin fiber.

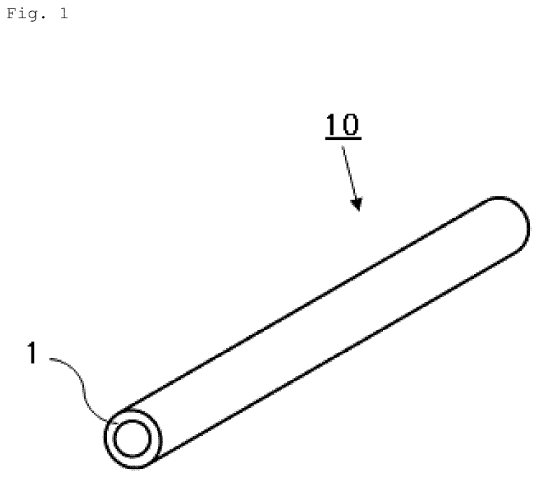

Now, the tube constituted by the tube wall (solely) made of a fiber means a cylindrical tube whose tube wall (circumferential wall of tube, or the wall surrounding the central cavity, denoted by reference sign 1 in FIG. 1) is solely made of a fiber. Such tube whose tube wall is solely made of a fiber, is a nonwoven fabric tube in which a fiber is deposited to form a nonwoven fabric.

The tube constituted by the tube wall that includes a fluororesin fiber deposited to form a nonwoven fabric, is different from a tube constituted by a stretched porous film of fluororesin having a node.

The inner diameter of the present tube is properly selectable depending on desired applications, which is preferably 1 to 30 mm, and more preferably 2 to 20 mm.

Also the length of the present tube is properly selectable depending on desired applications, which is preferably 1 to 30 cm, and more preferably 2 to 15 cm.

The thickness (wall thickness) of the tube wall of the present tube is properly selectable depending on desired applications, which is preferably 10 to 150 μm, more preferably 10 to 120 μm, and even more preferably 10 to 60 μm, from the viewpoint, for example, that the tube content is easily observable under water.

The present tube necessarily has air permeability, depending on applications (cell culture, for example). From the viewpoint of, for example, suitable use for such application, the present tube preferably has a Gurley permeability of 0.5 to 100 s, when measured perpendicular to the wall face, which is more preferably 1 to 80 s.

The Gurley permeability is specifically measured by a method described later in EXAMPLES.

Typically from the same reason described in relation with the Gurley permeability, the tube wall of the present tube preferably has a pore size (apparent pore size measured with use of a permporometer) of 0.05 to 10 μm, which is more preferably 0.1 to 3 μm.

Also typically from the same reason described in relation with the Gurley permeability, the tube wall of the present tube preferably has a porosity of 60% or larger, which is more preferably 80% or larger.

The pore size and porosity are specifically measured by a method described later in EXAMPLES.

The present tube allows observation (under an optical microscope, for example) of the tube content, which is a target to be observed placed or immobilized while using the cylindrical tube face or inside of the porous part as a scaffold, in an aqueous medium, thus enabling suitable use for applications where such observation is required, such as a culture tube or a culture medium for, for example, culturing microorganism or biological tissue (cell, for example).

The present tube also allows through-the-tube observation and monitoring of progress of a flow reaction that proceeds on the tube surface or inside the porous part, with use of, for example, a labeling substance, thus also enabling suitable use of the present tube as a reaction field (reactor) of the flow reaction.

The present tube is also suitably used as a separation tube (filter) that allows separation of a target substance, while observing the tube content that presents in a center cavity of the tube. The present tube may still also excel in suppressing adsorption of a biological substance (such as exosome) or a biopolymer (such as protein), thus enabling suitable use of the present tube as a separation tube (filter), for example, for producing a purified liquid with an increased purity of a target substance to be separated, from a sample liquid that contains such target substance to be separated (such as biological substance or biopolymer).

<Fluororesin Fiber>

The fluororesin fiber is not particularly limited as long as it is a fluororesin-containing fiber, and may be a fiber that contains any additive other than the fluororesin.

The fluororesin fiber used for the present tube may be one kind or two or more kinds.

The fluororesin is exemplified by, but not restrictively, polytetrafluoroethylene (PTFE), tetrafluoroethylene-perfluoroalkyl vinyl ether copolymer (PFA), tetrafluoroethylene-hexafluoropropylene copolymer (FEP), ethylene-tetrafluoroethylene copolymer (ETFE), tetrafluoroethylene-hexafluoropropylene-perfluoroalkyl vinyl ether copolymer (EPE), fluoroethylene-vinyl ether copolymer (FEVE), poly(chlorotrifluoroethylene) (PCTFE), ethylene-chlorotrifluoroethylene copolymer (ECTFE), polyvinylidene fluoride (PVDF), polyvinyl fluoride (PVF), vinylidene fluoride-hexafluoropropylene copolymer (VDF-HFP copolymer), and vinylidene fluoride-hexafluoropropylene-tetrafluoroethylene copolymer (VDF-HFP-TFE copolymer). Among them, PTFE and PFA are preferred, and PTFE is more preferred, from the viewpoint of, for example, enhanced exhibition of the effects of the present invention, and enhanced chemical resistance.

[Other Additives]

The fluororesin fiber may optionally contain, besides the fluororesin, any of known other additives having been blended to a fiber, without degrading the effects of the present invention. Such other additives are exemplified by polymer other than the fluororesin, anti-aging agent, antioxidant, stabilizer, silane coupling agent, filler (reinforcing material), plasticizer, flame retardant, waxes, and lubricant.

Only one kind of, or two or more kinds of such other additives may be used.

The filler is exemplified by functional fillers suited to applications of the present tube, and is typically exemplified by biocompatible filler in a case where the present tube is intended for use as a culture tube, or a culture medium for, for example, culturing microorganism or biological tissue (cell, for example). Such filler is specifically exemplified by inorganic fillers that include oxide ceramics (such as alumina, silica, zirconia, and titanium dioxide), apatitic ceramics (such as apatite, and hydroxyapatite), calcium phosphate ceramics (such as calcium phosphate, TCP [tricalcium phosphate], α-TCP, and calcium hydrogen phosphate), and glass materials (such as bioglass, crystallized glass, and silicate minerals (such as wollastonite)). Only one kind of, or two or more kinds of such fillers may be used.

The filler may have any shape not specifically limited, which is exemplified by particulate or fibrous shape.

The content of the filler in the fluororesin fiber, when contained therein, is preferably 0.01 to 50% by mass, which is more preferably 0.01 to 20% by mass, from the viewpoint of, for example, easiness of obtaining a fiber that demonstrates, for example, chemical resistance inherent to the fluororesin, and that fully demonstrates physical property inherent to the filler.

<Shape of Fluororesin Fiber>

The fluororesin fiber preferably has an average fiber diameter of 50 μm or shorter, which is more preferably 0.05 to 50 μm, even more preferably 0.1 to 20 μm, and particularly preferably 0.3 to 10 μm.

With the average fiber diameter fallen within the aforementioned ranges, the obtainable fluororesin tube can be highly flexible, and can advantageously have high distribution uniformity of fiber, even if formed to a fluororesin tube having the thickness (wall thickness) within the aforementioned ranges.

The average fiber diameter of the fluororesin fiber is controllable by properly selecting conditions for forming the fiber. In an exemplary case of electrospinning, the obtainable fiber tends to have reduced average fiber diameter, as a result of lowering the humidity during electrospinning, shrinking the nozzle diameter, increasing the applied voltage, or increasing the voltage density.

The average fiber diameter herein means an average value determined by observing the fiber (group) to be measured under a scanning electron microscope (SEM) (at 2000× magnification), randomly sampling 20 fibers on the obtained SEM image, measuring the diameter (long diameter) of the individual fibers, and averaging the measured results.

Coefficient of variation of fiber diameter of the fluororesin fiber, calculated by the equation below, is preferably 0.7 or smaller, and more preferably 0.01 to 0.5. With the coefficient of variation of fiber diameter controlled within the aforementioned range, meaning that the fiber diameter is uniform, the fluororesin tube that excels in mechanical strength is easily obtainable.

Coefficient of variation of fiber diameter=Standard deviation/Average fiber diameter

(Note “standard deviation” is derived from the fiber diameter of the aforementioned 20 fibers.)

The fluororesin fiber preferably, but not restrictively, has a fiber length of 0.1 to 1000 mm, which is more preferably 0.5 to 100 mm, and even more preferably 1 to 50 mm.

<Method for Manufacturing Fluororesin Fiber>
Spinning Step

The fluororesin fiber may be manufactured by, for example, electrospinning, melt spinning, melt electrospinning, spunbonding (meltblowing), wet spinning, or spunlacing. The fiber obtainable by electrospinning is particularly preferred.

The electrospinning, when employed for forming the fluororesin fiber, typically uses a spinning solution that contains the fluororesin and an optional solvent.

The proportion of the fluororesin contained in the spinning solution is typically 5 to 100% by mass, more preferably 5 to 80% by % by mass, and even more preferably 10 to 70% by mass.

Only one kind of, or two or more kinds of the fluororesin may be used.

The solvent is not particularly limited as long as it can dissolve or disperse therein the fluororesin, and is exemplified by water, dimethylacetamide, dimethylformamide, tetrahydrofuran, methylpyrrolidone, xylene, acetone, methyl ethyl ketone, chloroform, ethylbenzene, cyclohexane, benzene, sulfolane, methanol, ethanol, phenol, pyridine, propylene carbonate, acetonitrile, trichloroethane, hexafluoroisopropanol, and diethyl ether. Only one kind of, or two or more kinds of these solvents may be used.

The proportion of the solvent in the spinning solution is typically 0 to 90% by mass, more preferably 10 to 90% by mass, and even more preferably 20 to 80% by mass.

The spinning solution may further contain other components such as other additives which may be contained in the fluororesin fiber, surfactant, dispersant, charge control agent, adhesive, viscosity modifier, and fiber-forming agent. Only one kind of, or two or more kinds of each of such other components may be used.

When the fluororesin is poorly soluble to the solvent (for example, if the fluororesin is PTFE, and the solvent is water), the spinning solution preferably contains one kind of, or two or more kinds of fiber-forming agent, expecting easy retention of the fluororesin in a fiber shape during spinning.

The fiber-forming agent is preferably an organic polymer highly soluble to the solvent, and is exemplified by polyethylene oxide, polyethylene glycol, dextran, alginic acid, chitosan, starch, polyvinylpyrrolidone, polyacrylic acid, polymethacrylic acid, polyacrylamide, cellulose, and polyvinyl alcohol.

The content of the fiber-forming agent, when used, in the spinning solution is typically 0.1 to 15% by mass, and preferably 1 to 10% by mass, although depending on, for example, the viscosity of the solvent, or the solubility in the solvent.

When the fluororesin is PTFE, a preferred example of the spinning solution is a spinning solution (1) below.

Spinning solution (1): with PTFE content of 30% by mass or more and 70% by mass or less, preferably 35% by mass or more and preferably 60% by mass or less; content of fiber-forming agent of 0.1% by mass or more and 10% by mass or less, preferably 1% by mass or more and preferably 7% by mass or less; and the balance of solvent, totaling 100% by mass.

Conditions for the electrospinning may be exemplified as follows.

Applied voltage (applied voltage between a spinning nozzle and a fiber collector) is preferably 1 to 100 kV, more preferably 5 to 50 kV, and even more preferably 10 to 40 kV.

Spinning distance (distance between the spinning nozzle and the fiber collector) is preferably 5 to 30 cm.

The discharge speed of the spinning solution is preferably 0.01 to 3 mL/h.

The tip diameter (outer diameter) of the spinning nozzle used for electrospinning is preferably 0.1 to 2.0 mm, and more preferably 0.2 to 1.6 mm.

While the spinning atmosphere may be left uncontrolled, the spinning atmosphere preferably has a relative humidity of 10 to 50% for example, and a temperature of 10 to 35° C. for example.

More specifically, in an exemplary case where the aforementioned spinning solution (1) is employed, the applied voltage is preferably 10 to 50 kV, more preferably 10 to 40 kV, meanwhile the tip diameter (outer diameter) of the spinning nozzle is preferably 0.3 to 1.6 mm.
Firing Step

When the fluororesin is PTFE, the fluororesin is preferably subjected to a firing step after forming a fiber. The firing step may take place in step 2 below, or may succeed step 2.

The firing step is usually conducted by heat treatment at 200 to 390° C., for 10 to 300 minutes. The firing step can typically remove the solvent and the fiber-forming agent that possibly remain in the fiber.

<Hydrophilized Fluororesin Fiber>

Although the hydrophilized fluororesin fiber might be a fluororesin fiber obtainable by using a hydrophilic fluororesin as a raw material used for forming the fluororesin fiber, the hydrophilized fluororesin fiber is preferably a hydrophilized fluororesin fiber obtained by coating the fluororesin fiber obtained as described above, with a compound having a hydrophilic group (also referred to as “hydrophilic compound”, hereinafter), while considering, for example, easiness of obtaining the hydrophilic fluororesin fiber having desired hydrophilicity (average transmittance under water immersion, contact angle for water, for example).

The hydrophilic compound is specifically exemplified by hydrophilic compounds described later in relation with step 3, and a method for coating is exemplified by those described later in relation with step 3.

The hydrophilized fluororesin fiber is preferably a PTFE fiber obtained by coating the PTFE fiber with at least one polymer compound selected from polyvinyl alcohol and modified product thereof, polysaccharide and derivative thereof, collagen, gelatin, copolymer of vinyl alcohol and vinyl group-containing monomer, polyol, polyurethane, methacrylate and modified product thereof, and hydroxy group-containing (meth)acrylic polymer.

The polymer compound preferably has at least one functional group selected from hydroxy group, carboxylic acid group, sulfonic acid group, phosphonic acid group, ether group, epoxy group, amino group, amido group, and quaternary ammonium salt.

<<Method for Manufacturing Hydrophilic Fluororesin Tube>>

A method for manufacturing a hydrophilic fluororesin tube according to an embodiment of the present invention (hereinafter, also referred to as “the present method”) includes:

spinning step 1; spinning a fiber-forming material that contains at least one kind of fluororesin;

step 2; forming a fluororesin tube by depositing the fiber obtained in the step 1 onto a mandrel collector; and

step 3 hydrophilizing the fluororesin tube obtained in the step 2.

The present tube can be manufactured by the present method.

[Spinning Step 1]

The spinning step 1 is exemplified by a step same as the spinning step described in relation with the method for manufacturing the fluororesin fiber. Note that the fiber-forming material corresponds to the spinning solution used in the method for manufacturing the fluororesin fiber.

[Step 2]

The step 2 is a step for forming a fluororesin tube, by depositing the fiber obtained in the spinning step 1 on a mandrel collector.

The mandrel collector is a sort of collector designed to allow the fiber obtained in the spinning step 1 to deposit onto a mandrel that rotates at a predetermined speed, wherein the fiber obtained in the spinning step 1 deposits so as to surround the mandrel, thus forming a layer of the fiber in the form of nonwoven fabric around the mandrel. After thus forming the layer, the mandrel is drawn to leave the fluororesin tube.

The mandrel is exemplified by a metal rod (pipe), whose surface may be subjected to mold-releasing treatment.

The diameter (outer diameter) of the mandrel may be properly selected without special limitation depending on desired applications, and preferably such that the obtainable present tube will have the inner diameter within the aforementioned range.

The rotation speed of the mandrel is preferably 50 to 1000 rpm, and more preferably 100 to 500 rpm, from the viewpoint of, for example, easiness of obtaining the tube having high mechanical strength and desired shape.

The time the fiber obtained in the spinning step 1 deposits on the mandrel collector is preferably determined so that the obtainable present tube will have the thickness (wall thickness) within the aforementioned range.

The fluororesin fiber may be fired as described previously in relation with the method for manufacturing the fluororesin fiber, during the step 2, or after the step 2. In a particular case where the fluororesin is PTFE, the step 2 is preferably a step for forming the fluororesin tube, by depositing the fiber obtained in the step 1 on the mandrel collector, and then by firing the deposit.

The firing step is usually conducted by heat treatment at 200 to 390° C., for 10 to 300 minutes.

[Step 3]

The hydrophilic fluororesin tube might be manufactured by using, as the fiber-forming material used in the spinning step 1, a material that makes the obtainable fiber in the spinning step 1 demonstrate hydrophilicity. Meanwhile the present method has the step 3 for hydrophylizing the fluororesin tube obtained in the step 2, from the viewpoint of, for example, easiness of obtaining the hydrophilic fluororesin tube having desired hydrophilicity (average transmittance under water immersion, and contact angle for water, for example).

The step 3 is preferably a step for coating the fiber that constitutes the fluororesin tube obtained in the step 2, with a hydrophilic compound, and is more preferably the step 3′ described below. Specific examples of such step 3 are exemplified by steps described, for example, in WO 2014/021167 A, JP 2017-124350 A, and JP 2018-28011 A.

Step 3′: immersing the fluororesin tube obtained in the step 2 in a solution of a hydrophilic compound, thus coating the fiber that constitutes the fluororesin tube obtained in the step 2, with the hydrophilic compound, and then crosslinking the hydrophilic compound that coats the fiber.

The hydrophilic compound is not particularly limited as long as the effect of the present invention is not impaired, for which a compound having any shape, such as linear, branched, or dendrimer, may be used.

The hydrophilic compound is exemplified by hydroxy group-containing compound, carboxylic acid group-containing compound, sulfonic acid group-containing compound, phosphonic acid group-containing compound, ether group-containing compound, epoxy group-containing compound, amino group-containing compound, amido group-containing compound, and quaternary ammonium salt-containing compound.

Only one kind of, or two or more kinds of these compounds may be used.

By using at least one selected from hydroxy group-containing compound, carboxylic acid group-containing compound, sulfonic acid group-containing compound, ether group-containing compound, epoxy group-containing compound, amino group-containing compound, and amido group-containing compound as the hydrophilic compound, it becomes possible to easily obtain a hydrophilic fluororesin tube that excels in chemical resistance and is suitably used as a culture tube or a culture medium for, for example, culturing microorganism or biological tissue (cell, for example).

Also use of, as the hydrophilic compound, at least one selected from hydroxy group-containing compound, carboxylic acid group-containing compound, sulfonic acid group-containing compound, phosphonic acid group-containing compound, ether group-containing compound, epoxy group-containing compound, amino group-containing compound, amido group-containing compound, and quaternary ammonium salt-containing compound, makes it possible to obtain the hydrophilic fluororesin tube whose wall has at least one selected from hydroxy group, carboxylic acid group, sulfonic acid group, phosphonic acid group, ether group, epoxy group, amino group, amido group, and quaternary ammonium salt, and particularly makes it easier to obtain the hydrophilic fluororesin tube which is highly suppressive against adsorption of biological substance (such as exosome) and biological polymer (such as protein). Such hydrophilic fluororesin tube, which is highly suppressive against adsorption of biological substance and biological polymer, is suitably used as a separation tube (filter).

The hydroxy group-containing compound is exemplified typically, but not restrictively, by polyvinyl alcohol [PVA] and modified products thereof (such as ethylene oxide-modified PVA, carboxy group-modified PVA, sulfonic acid group-modified PVA, and quaternary ammonium-modified PVA); polysaccharides and derivatives thereof such as agarose, dextran, chitosan, cellulose and heparin; collagen; gelatin; copolymer of vinyl alcohol and vinyl group-containing monomer (such as vinyl alcohol-vinyl acetate copolymer, ethylene-vinyl alcohol copolymer, and vinyl alcohol-polyvinyl pyrrolidone copolymer); polyols such as (meth)acrylic polyol, fluorine-containing polyol, polyoxyalkylene (such as polyethylene glycol, copolymer of polyethylene glycol and polypropylene glycol [such as Pluronic F108 and Pluronic F127 (both from Sigma-Aldrich)]), polyester polyol, and diethylene glycol; and, hydroxy group-containing (meth)acrylic polymer such as copolymer that contains a crosslinked structure obtained by using hydroxy group-containing (meth)acrylic compound (such as 2-hydroxyethyl (meth)acrylate, 2-hydroxymethyl (meth)acrylate, 2-hydroxypropyl (meth)acrylate, and 2-hydroxybutyl (meth)acrylate), and di(meth)acrylic compound (such as tetraethylene glycol di(meth)acrylate).

The carboxylic acid group-containing compound is exemplified typically, but not restrictively, by copolymer of one kind, or two or more kinds of monomers selected from olefinic monomer such as ethylene, propylene and butylene, diene monomers such as butadiene, aromatic group-containing monomer such as styrene, and (meth)acrylate ester monomer such as acrylate ester and methacrylate ester, with a monomer having carboxylic acid group [—COOH] such as acrylic acid and methacrylic acid; homopolymer of monomer having carboxylic acid group such as acrylic acid and methacrylic acid; and carboxylic acid group-containing (meth)acrylic polymer such as copolymer that contains a crosslinked structure obtained by using carboxylic acid group-containing (meth)acrylic compound (such as 2-carboxyethyl (meth)acrylate, 2-carboxymethyl (meth)acrylate, 2-carboxypropyl (meth)acrylate, and 2-carboxybutyl (meth)acrylate), and di(meth)acrylic compound (such as tetraethylene glycol di(meth)acrylate); and amino acid.

The sulfonic acid group-containing compound is exemplified typically, but not restrictively, by copolymer of styrene and acrylamide-2-methylpropane sulfonic acid (salt); ternary copolymer of styrene, n-butyl acrylate, and acrylamide-2-methylpropane sulfonic acid (salt); ternary copolymer of styrene, 2-ethylhexyl acrylate, and acrylamide-2-methylpropane sulfonic acid (salt); and sulfonic acid group-containing (meth)acrylic polymer such as copolymer that contains a crosslinked structure obtained by using sulfonic acid group-containing (meth)acrylic compound (such as 2-sulfoethyl (meth)acrylate, 2-sulfomethyl (meth)acrylate, 2-sulfopropyl (meth)acrylate, and 2-sulfobutyl (meth)acrylate), and di(meth)acrylic compound (such as tetraethylene glycol di(meth)acrylate).

The phosphonic acid group-containing compound is exemplified typically, but not restrictively, by poly (meth)acryloyloxyethyl phosphorylcholine), and copolymer of (meth)acryloyloxyethyl phosphorylcholine with acrylic monomer.

In a case where phosphonic acid group-containing compound is used as the hydrophilic compound, first, a hydroxy group-containing compound, particularly PVA, is preferably used, followed by use of the phosphonic acid group-containing compound.

The ether group-containing compound is exemplified typically, but not restrictively, by polyethylene glycol and derivative thereof, fluororesin copolymer having ether group, polyurethane resin having ether group, and polyphenylene resin having ether group.

The epoxy group-containing compound is exemplified typically, but not restrictively, by epoxy resin, modified epoxy resin, acrylic (co)polymer having epoxy group, polybutadiene resin having epoxy group, polyurethane resin having epoxy group, and adduct or condensate of these resins.

The amino group-containing compound is exemplified typically, but not restrictively, by polyethyleneimine; polyvinylamine; polyamide polyamine; polyamidine; polydimethylaminoethyl methacrylate; polydimethylaminoethyl acrylate; and amino group-containing (meth)acrylic polymer such as copolymer that contains a crosslinked structure obtained by using amino group-containing (meth)acrylic compound (such as 2-aminoethyl (meth)acrylate, and 2-aminomethyl (meth)acrylate), and di(meth)acrylic compound (such as tetraethylene glycol di(meth)acrylate).

The amido group-containing compound is exemplified typically, but not restrictively, by poly(N-isopropyl (meth)acrylamide) and poly(N-vinyl-2-pyrrolidone).

The quaternary ammonium salt-containing compound is exemplified typically, but not restrictively, by saponified product of copolymer of vinyl ester, with quaternary ammonium salt-containing compound such as N-(meth)acrylamidomethyltrimethylammonium chloride, and allyltrimethylammonium chloride; and copolymer of (meth)acrylic monomer, with quaternary ammonium salt-containing compound such as N-(meth)acrylamidomethyltrimethylammonium chloride, and allyltrimethylammonium chloride.

The hydrophilic compound preferably, but not restrictively, has a weight average molecular weight of 100 to 1,000,000.

Among the hydrophilic compounds, preferred is a hydroxy group-containing compound, and more preferred are PVA, and hydroxy group-containing (meth)acrylic polymer.

The PVA preferably, but not restrictively, has a saponification value of 50 to 100%, and more preferably 60 to 100%, from the viewpoint of, for example, easiness of obtaining the present tube whose average transmittance under water immersion and contact angle for water fall within the aforementioned ranges.

The PVA also preferably, but not restrictively, has a weight average molecular weight of 200 to 150,000, and more preferably 500 to 100,000, from the viewpoint of, for example, fully immobilizing PVA onto the fiber that constitutes the fluororesin tube obtained in the step 2.

The concentration of PVA, when used as the hydrophilic compound, in the solution of the hydrophilic compound is preferably 0.1 to 1.5% by mass, and more preferably 0.1 to 1.0% by mass.

Use of the solution, whose PVA concentration falls within the aforementioned range, makes it possible to easily obtain the present tube having the average transmittance under water immersion and the contact angle for water within the aforementioned ranges, and thus being highly hydrophilic. This is preferred also since the fluororesin tube obtained in the step 2 is less likely to cause clogging of pores that reside on the tube wall.

In an alternative case where the hydroxy group-containing (meth)acrylic polymer is used as the hydrophilic compound, the concentration of the hydrophilic monomer that constitutes the hydroxy group-containing (meth)acrylic polymer, in the solution of the hydrophilic compound, is preferably 1 to 20% by mass.

Use of the solution, whose hydrophilic monomer concentration falls within the aforementioned range, makes it possible to easily obtain the present tube having the average transmittance under water immersion and the contact angle for water within the aforementioned ranges, and thus being highly hydrophilic. This is preferred also since the fluororesin tube obtained in the step 2 is less likely to cause clogging of pores that reside on the tube wall.

When the hydrophilic compound used herein is not liquid, the solution of the hydrophilic compound preferably uses a solvent that can dissolve the compound.

The solvent may be properly selected without special limitation depending on the hydrophilic compound to be used, and is specifically exemplified by water; alcohols such as methanol, ethanol, n-propanol, isopropanol (IPA), n-butanol, sec-butanol, tert-butanol, and isobutanol; esters such as methyl acetate, ethyl acetate, and butyl acetate; ketones such as acetone and methyl ethyl ketone; aromatic hydrocarbons such as toluene and xylene; and ethers such as diethyl ether, dibutyl ether, tetrahydrofuran, and dioxane. Among them, water is preferred.

Only one kind of, or two or more kinds of these solvent may be used.

The time the fluororesin tube obtained in the step 2 is immersed in the solution of the hydrophilic compound is not particularly limited as long as the fiber that constitutes the fluororesin tube obtained in the step 2 can be coated with the hydrophilic compound, and is preferably 10 seconds to 180 minutes, and more preferably 30 seconds to 60 minutes, typically depending on the concentration of the hydrophilic compound to be used, in the solution of such hydrophilic compound.

The temperature and atmosphere of immersion is properly selectable without special limitation, typically depending on type of the hydrophilic compound.

Note in a case where an aqueous solution is used as the solution of the hydrophilic compound, the hydrophilic compound tends to poorly permeate deep inside the fiber that constitutes the fluororesin tube (the tube obtained in the aforementioned step 2) when immersed in such aqueous solution of the hydrophilic compound, when the fluororesin tube (the fluororesin tube obtained in the step 2) is left untreated. The fluororesin tube obtained in the step 2 is therefore preferably immersed in a solvent compatible with water such as isopropyl alcohol (that is, the solvent compatible with water is impregnated into the fluororesin tube obtained in the step 2), before being immersed in the solution of the hydrophilic compound.

In a case where the fluororesin tube obtained in the step 2 is immersed in a solvent compatible with water, the fluororesin tube having been immersed in the solvent compatible with water is preferably taken out from the solvent, and is then preferably immersed in a solution of the hydrophilic compound, to replace the solvent compatible with water with the hydrophilic compound. In this case, mechanical load may be applied to the fluororesin tube to embed the hydrophilic compound into the fluororesin tube. More specifically, the hydrophilic compound may be made easier to permeate into the fluororesin tube, by press-rubbing, or under depressurizing or pressurizing with use of a vacuum/pressure impregnation apparatus.

The solvent compatible with water is preferably, but not restrictively, easy to permeate into the fluororesin and easy to volatilize, and is specifically exemplified by alcohols such as methanol, ethanol, n-propanol, isopropanol, n-butanol, sec-butanol, tert-butanol, and isobutanol; esters such as methyl acetate, ethyl acetate, and butyl acetate; ketones such as acetone and methyl ethyl ketone; ethers such as tetrahydrofuran and dioxane; and aprotic polar solvents such as dimethylsulfoxide and N,N-dimethylformamide. Among them, isopropanol is preferred, from the viewpoint of, for example, easiness of permeation into the fluororesin.

The solvent compatible with water may be a liquid obtainable by mixing at least one selected from the aforementioned alcohols, esters, ketones, ethers, or aprotic polar solvents, with water.

Only one kind of, or two or more kinds of these solvents compatible with water may be used.

The time the fluororesin tube obtained in the step 2 is immersed in the solvent compatible with water is typically, but not restrictively, 1 minute to 24 hours.

The temperature and atmosphere of immersion are not particularly limited.

Method for crosslinking the hydrophilic compound is exemplified by irradiation crosslinking with use of ionizing radiation such as electron beam, thermal crosslinking, and chemical crosslinking with use of crosslinking agent. Among these crosslinking methods, chemical crosslinking with use of crosslinking agent is preferred, since, for example, the crosslinking can proceed also in aqueous solution in a reliable manner.

The crosslinking agent usable for the chemical crosslinking is properly selectable depending on the type of hydrophilic compound to be used, without special limitation, and is exemplified by aldehyde compounds such as formaldehyde, glutaraldehyde, and terephthalaldehyde; ketone compounds such as diacetyl and chloropentanedione; compound having reactive halogen such as bis(2-chloroethylurea)-2-hydroxy-4,6-dichloro-1,3,5-triazine; compound having reactive olefin such as divinylsulfone; N-methylol compound; isocyanates; aziridine compounds; carbodiimide compounds; epoxy compounds; halogenated carboxyaldehydes such as mucochloric acid; dioxane derivatives such as dihydroxydioxane; inorganic crosslinking agents such as chromium alum, zirconium sulfate, boric acid, borate, and phosphate; diazo compound such as 1,1-bis(diazoacetyl)-2-phenylethane; compound that contains disuccinimidyl ester; and bifunctional maleimides.

Only one kind of, or two or more kinds of these crosslinking agents may be used.

The crosslinking agent is preferably used in large excess over the content of the hydrophilic compound used in the solution.

Among the crosslinking methods, the chemical crosslinking conducted with use of the aldehyde compound such as glutaraldehyde or terephthalaldehyde, in the presence of an acid catalyst, is particularly preferred, since reactivity at normal temperature is high, degree of crosslinkage is easily stable at a prescribed quantity, the method is less likely to be affected by alcohol during crosslinking, and the acetal bond which is a cross-linking point produced has relatively high chemical resistance.

The acid catalyst may be any of known catalysts without special limitation, and is exemplified by hydrochloric acid.

The amount of consumption of the acid catalyst is preferably 0.1 to 10% by mass, per 100% by mass of the crosslinking agent used herein.

The temperature during the crosslinking is preferably 10 to 95° C., from the viewpoint of, for example, easiness of obtaining the present tube that excels in chemical resistance, which is more preferably 20 to 60° C.

The crosslinking time for the crosslinking is preferably 1 to 60 minutes, from the viewpoint of, for example, durability of the obtained present tube, and productivity.

The fluororesin tube after went through the step 3 is preferably washed typically with water, principally for the purpose of, for example, removing any unreacted hydrophilic compound.

The step of washing may take place under heating as necessary.

### EXAMPLES

Next, an embodiment of the present invention will further be detailed with reference to Examples, to which the present invention is by no means limited.

[Exemplary Preparation]

Polyethylene oxide (PEO18, from Sumitomo Seika Chemicals Co., Ltd., molecular weight: 300,000), ethanol (special grade, from FUJIFILM Wako Pure Chemical Corporation), and water were mixed, and stirred for one day, to obtain a mixture.

The obtained mixture and a PTFE dispersion (D210-C, from DAIKIN Industries, Ltd., PTFE content: 60% by mass) were mixed under stirring for one hour with use of a planetary centrifugal mixer, to prepare a spinning solution (fiber-forming material).

The PTFE content in the obtained spinning solution was 45 parts by mass, the polyethylene oxide content was 1 part by mass, the ethanol content was 3.3 parts by mass, and the water content was 49 parts by mass.

### Example 1

A fiber was formed from the thus prepared spinning solution, with use of electrospinning system NANON-03 (from MECC Co., Ltd.), and deposited on a mandrel (SUS pipe, diameter: φ6 mm, length: 170 mm) of a mandrel collector (C-MA, from MECC Co., Ltd.), under the following conditions.
Needle diameter: 27 G
Spinneret width: 130 mm (tube length)
Applied voltage: 15 kV
Liquid feeding rate: 0.5 mL/h
Spinning time: 15 min
Rotating speed of mandrel: 300 rpm

The fiber deposited on the mandrel was pre-dried at 80° C. for 3 hours, then heated to 240° C., and kept for a certain period of time. The fiber was then fired at 360° C. for 15 minutes, and then cooled down to room temperature, to manufacture a 100 mm long fluororesin tube. Thereafter, the fluororesin tube was detached from the mandrel.

A part of the obtained fluororesin tube was cut, and subjected to measurement of the wall thickness (thickness of the tube wall). The tube wall was 50 μm thick.

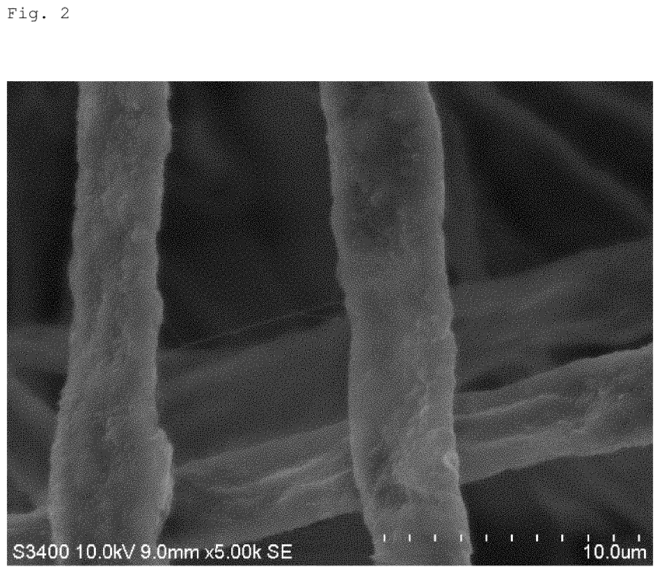

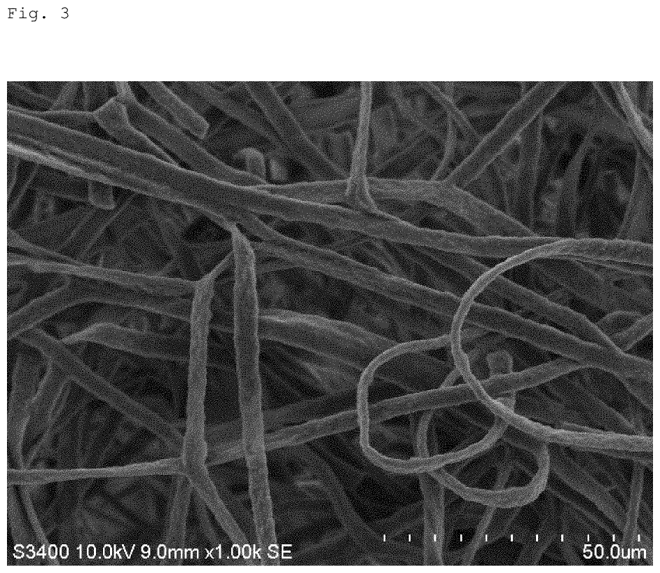

A part of the obtained fluororesin tube was cut, and observed under a SEM (S-3400N, from Hitachi High-Technologies Corporation) at 5000× magnification and 1000× magnification. FIG. 2 is an SEM image of the obtained fluororesin tube at 5000× magnification, and FIG. 3 is an SEM image of the obtained fluororesin tube at 1000× magnification.

From these SEM images, the obtained fluororesin tube was found to be a tube made of a nonwoven fabric of PTFE fiber, and the PTFE fiber that constitutes the obtained fluororesin tube was found to have a fiber diameter of approximately 3 μm.

The obtained fluororesin tube was immersed in a 99.7% isopropyl alcohol (IPA) solution (from FUJIFILM Wako Pure Chemical Corporation) for one minute at room temperature (25° C.)

Next, the fluororesin tube was taken out from the IPA solution, and immersed at room temperature for 10 minutes, in 500 mL of an aqueous solution of polyvinyl alcohol (PVA) (from FUJIFILM Wako Pure Chemical Corporation, 160-11485, degree of polymerization: 1500, degree of saponification: 98%) with the concentration adjusted to 0.5% by mass.

The fluororesin tube was then taken out from the PVA solution, and immersed at room temperature for 60 minutes, in a mixed liquid obtained by mixing 500 mL of a 5% by mass glutaraldehyde solution (obtained by diluting a 25% glutaraldehyde solution from FUJIFILM Wako Pure Chemical Corporation with pure water, to adjust the concentration to 5% by mass), and 5 mL of 36% hydrochloric acid (from FUJIFILM Wako Pure Chemical Corporation).

The fluororesin tube was then taken out from the mixed liquid, placed in pure water, and boiled at 95° C. for 30 minutes, thereby dissolving unreacted IPA, PVA, and glutaraldehyde.

After the boiling, the fluororesin tube was taken out from the liquid, and was allowed to naturally dry, to manufacture a hydrophilic fluororesin tube.

The tube wall of the thus obtained hydrophilic fluororesin tube was sampled from anywhere at three points, and then subjected to measurement of thickness (wall thickness of the tube wall). An average value of the thickness of the tube walls, sampled at three points, was 50 μm (also the thickness of the tube walls of the hydrophilic fluororesin tubes described below was measured in the same way).

Observation of the SEM image of the obtained hydrophilic fluororesin tube, conducted in the same way as described previously, also revealed that the fiber diameter of the hydrophilized PTFE fiber that constitutes the obtained hydrophilic fluororesin tube was approximately 3 μm (also the fiber diameter of the hydrophilized PTFE fibers that constitute the following hydrophilic fluororesin tubes was measured in the same manner).

### Example 2

A hydrophilic fluororesin tube was manufactured in the same manner as in Example 1, except that the liquid feeding rate was changed to 1 mL/h, and the spinning time was changed to 30 minutes.

The thickness of the tube wall of the obtained hydrophilic fluororesin tube was 100 μm, and the fiber diameter of the hydrophilized PTFE fiber that constitutes the obtained hydrophilic fluororesin tube was approximately 2 μm.

### Example 3

A fluororesin tube obtained in the same manner as in Example 1 (the fluororesin tube before immersed in the IPA solution in Example 1) was immersed in a 99.7% isopropyl alcohol (IPA) solution (from FUJIFILM Wako Pure Chemical Corporation), at room temperature (25° C.) for one minute.

Next, the fluororesin tube was taken out from the IPA solution, and then immersed at 65° C. for one hour, in an aqueous solution prepared by dissolving 7 g of hydroxypropyl methacrylate (mixture of 2-hydroxypropyl ester and 2-hydroxy-1-methylethyl ester) (HPA) (from Tokyo Chemical Industry Co., Ltd., product code: M0512), 4 g of tetraethylene glycol diacrylate (TEGDA) (from Tokyo Chemical Industry Co., Ltd., product code: T1569), and 1 g of ammonium persulfate (AmPS) (from Tokyo Chemical Industry Co., Ltd., product code: A2098), into 100 g of pure water.

The fluororesin tube was then taken out from the aqueous solution, placed in pure water, and boiled at 85° C. for 6 hours, thereby dissolving unreacted HPA, TEGDA, and AmPS.

After the boiling, the fluororesin tube was taken out from the liquid, and was allowed to naturally dry, to manufacture a hydrophilic fluororesin tube.

The thickness of the tube wall of the obtained hydrophilic fluororesin tube was 50 μm, and the fiber diameter of the hydrophilized PTFE fiber that constitutes the obtained hydrophilic fluororesin tube was approximately 3 μm.

### Example 4

A hydrophilic fluororesin tube was prepared in the same manner as in Example 3, except that the aqueous solution, in which the fluororesin tube after being taken out from the IPA solution is immersed, was changed to an aqueous solution prepared by dissolving 7 g of hydroxypropyl methacrylate (a mixture of 2-hydroxypropyl ester and 2-hydroxy-1-methylethyl ester, from Tokyo Chemical Industry Co., Ltd., product code: M0512), 0.7 g of tetraethylene glycol diacrylate (from Tokyo Chemical Industry Co., Ltd., product code: T1569), and 1.3 g of ammonium persulfate (from Tokyo Chemical Industry Co., Ltd., product code: A2098), into 100 g of pure water.

The thickness of the tube wall of the obtained hydrophilic fluororesin tube was 50 μm, and the fiber diameter of the hydrophilized PTFE fiber that constitutes the obtained hydrophilic fluororesin tube was approximately 3 μm.

The obtained hydrophilic fluororesin tube was confirmed to have a suppressive effect on exosome adsorption.

### Example 5

A PTFE nonwoven fabric sheet made of ultrafine PTFE fiber (basis weight: 24 g/m², size: 200 mm×200 mm×54 μm, average pore size: 1.9 μm, fiber diameter: 1 μm) was immersed in a 99.7% isopropyl alcohol (IPA) solution (from FUJIFILM Wako Pure Chemical Corporation), at room temperature (25° C.) for one minute.

Next, the PTFE nonwoven fabric sheet was taken out from the IPA solution, and immersed at room temperature for 30 minutes, in 500 mL of an aqueous solution of polyvinyl alcohol (PVA) (from FUJIFILM Wako Pure Chemical Corporation, 160-11485, degree of polymerization: 1500, degree of saponification: 98%) with the concentration adjusted to 0.1% by mass.

The PTFE nonwoven fabric was then taken out from the PVA solution, and immersed at room temperature for 30 minutes, in a mixed solution of 500 mL of a 5% by mass glutaraldehyde solution (a pure water dilution of a 25% glutaraldehyde solution from FUJIFILM Wako Pure Chemical Corporation, with the concentration adjusted to 5% by mass), and 5 mL of 36% hydrochloric acid (from FUJIFILM Wako Pure Chemical Corporation).

The PTFE nonwoven fabric was then taken out from the mixed solution, and placed in pure water, thereby dissolving unreacted IPA, PVA, and glutaraldehyde.

The PTFE nonwoven fabric was then taken out from the solution, and allowed to naturally dry, to obtain a hydrophilized PTFE nonwoven fabric sheet.

The obtained hydrophilized PTFE nonwoven fabric sheet was further immersed at room temperature (25° C.) for 30 minutes, in an ethanol solution of 2-methacryloyloxyethyl phosphorylcholine (MPC) polymer (MP011 L1 from Intelligent Surfaces Inc.).

The sheet was then taken out from the ethanol solution, allowed to naturally dry, washed with pure water, and again allowed to naturally dry, to manufacture a hydrophilic fluororesin sheet. The thus manufactured hydrophilic fluororesin sheet was formed into a cylindrical shape and partially fixed, to manufacture a hydrophilic fluororesin tube.

The thickness of the tube wall of the obtained hydrophilic fluororesin tube was 54 μm, and the diameter of the hydrophilized PTFE fiber that constitutes the obtained hydrophilic fluororesin tube was approximately 1 μm.

### Comparative Example 1

A hydrophilized stretched PTFE membrane (product number: Omnipore JAWP04700, from Millipore K.K., pore size: 1.0 μm) was used for Comparative Example 1.

<<Tests>>

The tests below were conducted, with use of three test pieces, sampled anywhere at three points from the tube wall of each of the hydrophilic fluororesin tubes obtained in Examples 1 to 5, and three test pieces sampled anywhere at three points from the stretched PTFE membrane used in Comparative Example 1.

<Average Transmittance Under Water Immersion>

Each test piece was placed on the inner wall of a 10 mm square measurement cell made of quartz glass, and the cell was filled with pure water. One minute after the filling with pure water, the transmittance (transmittance of light perpendicularly incident on the wall face and the test piece) was measured at 10 nm wavelength intervals from 400 to 700 nm wavelength, with use of an ultraviolet-visible spectrophotometer (UV-2600, from Shimadzu Corporation), and average transmittance (average transmittance under water immersion) of the three test pieces was calculated. The results are summarized in Table 1.

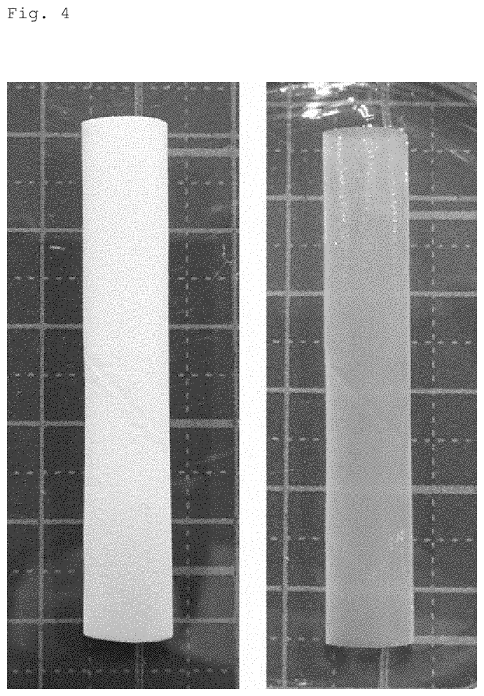

A photograph of an appearance of the hydrophilic fluororesin tube obtained in Example 1 before such immersion in water was shown in FIG. 4-left, and a photograph of an appearance immediately after such immersion in water was shown in FIG. 4-right.

<Contact Angle for Water>

Each of the three test pieces was placed on a stage whose plane is perpendicular to the gravity.

Onto each of the three test pieces thus placed on the stage, 1 μL of water was added dropwise at 25° C., and 10 seconds after the dropwise addition, the contact angle for water was measured with use of a contact angle meter (DMo-601, from Kyowa Interface Science Co., Ltd.), and the average value thereof was calculated. The results are summarized in Table 1.

<Gurley Permeability>

Gurley permeability of each of the three test pieces (air permeability in the direction perpendicular to the principal face of each test piece) was measured with use of a through-pore size distribution measuring apparatus (Permporometer CFP-1200-AEL, from PMI), and the average value was calculated. The results are summarized in Table 1.

<Pore Size>

Pore size (average pore size) of each of the three test pieces was measured with use of a through-pore size distribution measuring apparatus (CFP-1200-AEL, from PMI), and the average value was calculated. The results are summarized in Table 1.

<Porosity>

The mass and density of each of the three test pieces were measured, the porosity of the test piece was measured from these values, and the average value was calculated. The results are summarized in Table 1.

TABLE 1

| | Average | | | | | |
|---|---|---|---|---|---|---|
| | transmittance | Wall | Contact | Gurley | | |
| | under water | thick- | angle for | perme- | Pore | |
| | immersion | ness | water | ability | size | Porosity |
| | (%) | (μm) | (°) | (sec) | (μm) | (%) |
| Example 1 | 67.4 | 50 | 0 | 2 | 1.8 | 85 |
| Example 2 | 41.4 | 100 | 0 | 4 | 1.5 | 84 |
| Example 3 | 57.0 | 50 | 0 | 6 | 1.6 | 84 |
| Example 4 | 56.9 | 50 | 0 | 6 | 1.6 | 84 |
| Example 5 | 66.8 | 54 | 0 | 5 | 1.9 | 81 |
| Comparative | 39.8 | 50 | 0 | 2.3 | 1.0 | 86 |
| Example 1 | | | | | | |

<Chemical Resistance>

A test piece, sampled anywhere at one point from each of the hydrophilic fluororesin tube obtained in Examples 1 to 4, was immersed in each of the following solvents at 25° C. for one day and 7 days. The test piece each after immersion for one day and 7 days was taken out, washed with water, and then dried. Water was dropped on the dried test piece to evaluate wettability. The wettability was evaluated at 25° C. with use of a contact angle meter (DMo-601, from manufactured by Kyowa Interface Science Co., Ltd.), with which 1 μL of water was dropped. The wettability was rated as 0 (excellent resistance (chemical resistance) against the solvent), when the contact angle was found to become 0° within a second. The results are summarized in Table 2.
Aqueous HCl solution: pure water dilution of 36% hydrochloric acid (from FUJIFILM Wako Pure Chemical Corporation, special grade), with the HCl concentration adjusted to 1.5% by mass (pH=1).
Aqueous NaOH solution: aqueous solution prepared by dissolving 4 g of sodium hydroxide in 100 g of pure water (NaOH concentration: 1 M, pH=14).
Aqueous ethanol solution: pure water dilution of 99.5% ethanol (DMSO, from FUJIFILM Wako Pure Chemical Corporation, special grade), with the ethanol concentration adjusted to 50% by mass.
Aqueous DMSO solution: pure water dilution of dimethylformamide (from FUJIFILM Wako Pure Chemical Corporation, special grade), with the DMSO concentration adjusted to 50% by mass.

TABLE 2

| | | | Aq. HCl | Aq. NaOH | | |
|---|---|---|---|---|---|---|
| | | | solution | solution | | |
| | | | 1.5% | 1M | Ethanol | DMSO |
| | Immersion time | | (pH = 1) | (pH = 14) | 50% | 50% |
| Example 1 | 1 | day | ◯ | ◯ | ◯ | ◯ |
| | 7 | days | ◯ | ◯ | ◯ | ◯ |
| Example 2 | 1 | day | ◯ | ◯ | ◯ | ◯ |
| | 7 | days | ◯ | ◯ | ◯ | ◯ |
| Example 3 | 1 | day | ◯ | ◯ | ◯ | ◯ |
| | 7 | days | ◯ | ◯ | ◯ | ◯ |
| Example 4 | 1 | day | ◯ | ◯ | ◯ | ◯ |
| | 7 | days | ◯ | ◯ | ◯ | ◯ |

### REFERENCE SIGNS LIST

1 Tube wall
10 Tube (hydrophilic fluororesin tube)

What is claimed is:

1. A hydrophilic fluororesin tube
comprising a tube wall that comprises a fluororesin fiber deposited to form a nonwoven fabric, and
satisfying requirement (1):
requirement (1); the tube wall of the hydrophilic fluororesin tube demonstrating an average transmittance, when immersed in water at 25° C. for 1 minute, of 40% or larger at 400 to 700 nm wavelength.

2. The hydrophilic fluororesin tube according to claim 1, wherein the tube wall has a thickness of 10 to 150 μm.

3. The hydrophilic fluororesin tube according to claim 1, wherein the tube wall has a Gurley permeability of 0.5 to 100 s, when measured in a direction perpendicular to the wall face.

4. The hydrophilic fluororesin tube according to claim 1, comprising the tube wall that comprises a polytetrafluoroethylene fiber.

5. The hydrophilic fluororesin tube according to claim 4, wherein the polytetrafluoroethylene fiber is a polytetrafluoroethylene fiber coated with at least one polymer compound selected from polyvinyl alcohol and modified product thereof, polysaccharide and derivative thereof, collagen, gelatin, copolymer of vinyl alcohol and vinyl group-containing monomer, polyol, polyurethane, methacrylate and modified product thereof, and hydroxy group-containing (meth)acrylic polymer.

6. The hydrophilic fluororesin tube according to claim 5, wherein the polymer compound has at least one functional group selected from hydroxy group, carboxylic acid group, sulfonic acid group, phosphonic acid group, ether group, epoxy group, amino group, amido group, and quaternary ammonium salt.

7. A method for manufacturing a hydrophilic fluororesin tube, comprising:
spinning a fiber-forming material that comprises at least one kind of fluororesin;
forming a fluororesin tube by depositing the fiber obtained in the spinning a fiber-forming material onto a mandrel collector; and
hydrophilizing the fluororesin tube obtained in the forming a fluororesin tube.

8. The method for manufacturing a hydrophilic fluororesin tube according to claim 7, wherein
the fluororesin is polytetrafluoroethylene,
the forming a fluororesin tube is forming a fluororesin tube by depositing the fiber obtained in spinning a fiber-forming material, and then by firing the deposit.
